(19) United States
(12) Patent Application Publication
(10) Pub. No.: US 2008/0000472 A1
(43) Pub. Date: Jan. 3, 2008

(54) RESPIRATORY THERAPY APPARATUS WITH OXYGEN FLOW DIVERTER

(76) Inventor: Nancy Wall, Grandville, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/755,088

(22) Filed: May 30, 2007

Related U.S. Application Data
(60) Provisional application No. 60/818,214, filed on Jul. 3, 2006.

(51) Int. Cl. A61M 16/20

(52) U.S. Cl. 128/202.27; 128/205.24

(57) ABSTRACT

A three-way diverter valve has a body with an input with a fitting for securing the input to the output of a flowmeter. The valve body also includes first and second outputs and a ball coupled to a control handle for rotating the ball valve to first or second positions. The ball has a passageway for supplying oxygen to either said first output or said second output. The control handle may include indicia identifying the active flow direction for the selected output.

### CROSS-REFERENCE TO RELATED APPLICATIONS

This application claims priority under 35 U.S.C. § 119(e) on U.S. Provisional Application No. 60/818,214 entitled OXYGEN FLOW DIVERTER, filed on Jul. 3, 2006, by Nancy Myers Wall, the entire disclosure of which is incorporated herein by reference.

### BACKGROUND OF THE INVENTION

The present invention relates to an apparatus for use in a hospital for treatment of respiratory diseases and particularly to a system for allowing a therapist to easily treat patients requiring either supplemental oxygen for breathing or for small volume nebulizer treatments without the need for disconnecting and reconnecting various fittings.

Statistics show that the occurrence of respiratory ailments is dramatically increasing in the U.S. population. Such ailments include asthma and chronic obstructive pulmonary disease (COPD), and secondary respiratory problems caused by pneumonia and bronchitis. These maladies frequently require the patient to be hospitalized and/or visit a treatment center to be provided with supplemental oxygen to ease their breathing. Such oxygen is typically provided to a patient through a cannula or, in some cases, a breathing mask. Frequently, the patient, if breathing becomes acutely difficult or if ordered on a scheduled basis, receives a small volume nebulizer treatment in which a bronchial dilator albuterol medication, such as Proventil®, is entrained through a small volume nebulizer to assist in clearing the airways of the patient to facilitate the exchange of oxygen by the lungs.

In hospitals, oxygen is supplied to rooms from a central source at approximately 50 psi (pounds per square inch). A flowmeter is secured by a fitting to a wall outlet in each of the hospital rooms and can be adjusted to control the flow rate to an outlet to which there is attached either a cannula or oxygen mask, frequently through a container of humidifying water to provide oxygen at a flow rate of typically about 2-3 liters/minute (lpm). When a nebulizer treatment is necessary, the respiratory therapist must shut off the flowmeter, disconnect the coupling to the cannula or mask, and subsequently reattach a conduit from the nebulizer unit to the flowmeter outlet. The flowmeter is then turned on and adjusted to a flow rate of about 6-8 liters/minute to supply oxygen to the nebulizer unit. The nebulizer unit supplies a medicament to the patient either through a mask or nebulizer tube. In view of the fact that numerous treatments are given throughout the day to many patients, the disconnecting and reconnecting of the various tubing requires a significant amount of the therapist's time and, in some cases, can delay necessary emergency nebulizing treatment to a patient.

There exists a need, therefore, for a system which facilitates the treatment of respiratory patients and provides a greater efficiency to the therapist in providing a supply of oxygen both for assisted breathing and for a therapeutic nebulizing treatment.

### SUMMARY OF THE INVENTION

The system of the present invention overcomes the difficulties of the prior art by providing a treatment apparatus including a three-way diverter valve which has an input adapted to be coupled to the output of an oxygen flowmeter and two outputs which can be sequentially selected utilizing a control handle. The three-way valve has a first output which can be coupled to a cannula or to a cannula through a humidifying container of water which is selected by a first position of the valve. The valve has a second output which, upon rotating the control handle of the valve, diverts the oxygen to the second output to which a nebulizer treatment tube can be coupled, diverting the flow of oxygen from the cannula to the nebulizer treatment tube. Both the cannula and the nebulizer tube may, therefore, be continuously attached to the apparatus and allows the respiratory therapist to easily change from one mode of operation to the other without requiring disconnecting and reconnecting a variety of fittings. This greatly improves the efficiency of the therapist's time and improves patient safety by allowing rapid access to nebulizing treatments if necessary on an acute basis.

In a preferred embodiment of the invention, the apparatus comprises a flow diverter valve having a body with an input with a fitting for securing the input to the output of a flowmeter. The valve body also includes first and second outputs and a ball coupled to a control handle for rotating the ball valve to first or second positions. The ball has a passageway for supplying oxygen to either said first output or said second output. The control handle may include indicia identifying the active flow direction for the selected output.

Thus, with the apparatus of the present invention, the respiratory therapist can more efficiently tend to patient care and needs and, as a result, can handle a greater number of patients during a given time period, thereby reducing overall patient cost. Also, patient safety and care is improved by the substantially faster availability of emergency nebulizer treatments.

These and other features, objects and advantages of the present invention will become apparent upon reading the following description thereof together with reference to the accompanying drawings.

### BRIEF DESCRIPTION OF THE DRAWINGS

FIG. 1 is a perspective view of the apparatus embodying the present invention, showing various equipment coupled to an oxygen supply flowmeter;

FIG. 2 is an exploded perspective view of the positioning of the oxygen flow apparatus of the present invention;

FIG. 3 is a front elevational view, partly in phantom form, of the oxygen flow diverter valve incorporating the present invention shown in the first position; and

FIG. 4 is a front elevational view, partly in phantom form, of the oxygen flow diverter valve incorporating the present invention shown in the second position.

### DETAILED DESCRIPTION OF THE PREFERRED EMBODIMENT

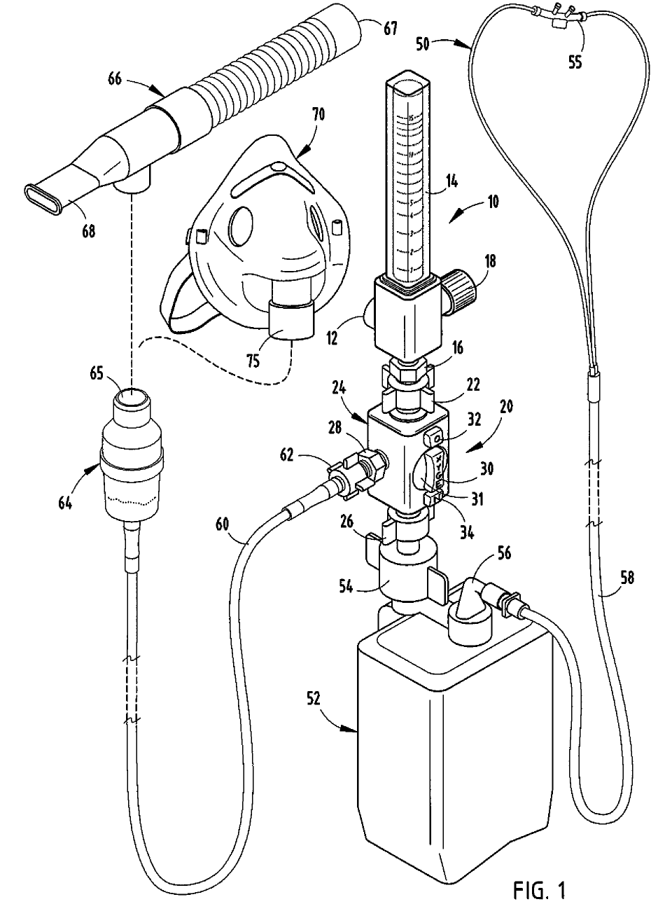

FIG. 1 shows an installation of the invention in a hospital or other facility in which respiratory treatment is required for patients. The system typically will include a flowmeter 10, which has a rear fitting 12 which attaches to an oxygen outlet in the wall of a facility. The flowmeter includes a ball-type flow gage 14 indicating the liters per minute flow of oxygen at an outlet 16. The flow rate is adjustable by a valve control handle 18 to select the flow rate of oxygen supplied to either a breathing cannula 50 or a nebulizer tube 60 or to shut off the oxygen.

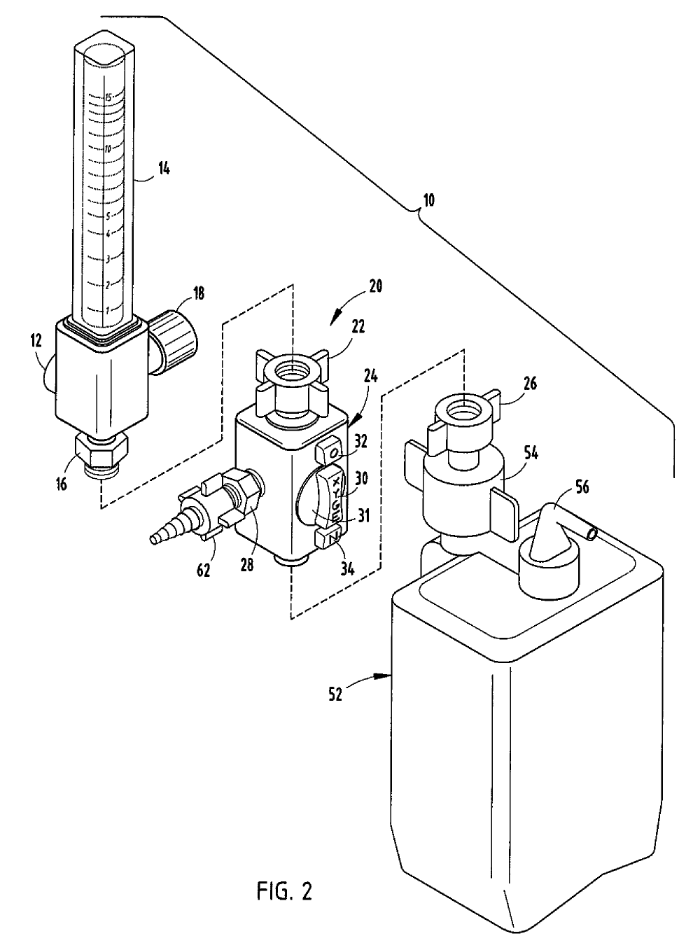

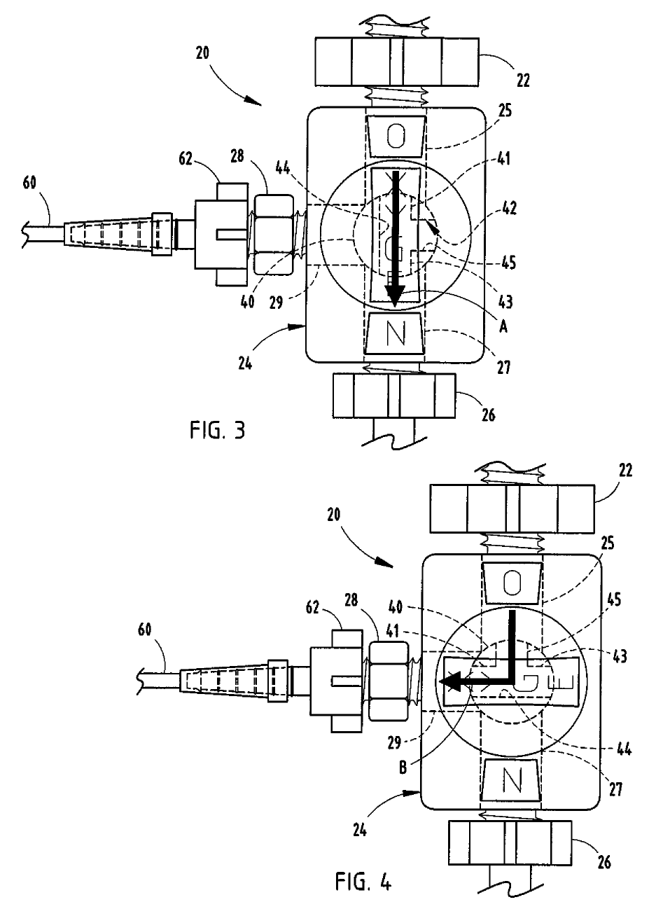

The outlet 16 of flowmeter 10 includes a threaded end which receives a rotatable, flanged, internally threaded coupling 22 of a flow diverter valve 20 of the present invention. Coupling 22 allows the valve to be sealably attached to the outlet 16 of the flowmeter. Valve 20 includes a valve body 24, an oxygen inlet 25 (FIGS. 3 and 4) to which coupling 22 is threadably secured. Valve 20 also includes a first outlet 27 (FIGS. 3 and 4) with a fitting 26 threadably coupled thereto for coupling to fitting 54 of a humidifying tank 52 of a cannula unit. Valve 20 also includes a second outlet 29 (FIGS. 3 and 4), which is coupled by a threaded fitting 28 to receive an adapter nipple 62 to which a nebulizer tube 60 can be attached. Valve 20 is a three-way ball valve with a control handle 30 which may include a raised center section, as best seen in FIGS. 1 and 2, which includes indicia 31 either embossed, debossed, or printed thereon, which integrates with indicia 32 and 34 on valve body 24 to indicate the direction of flow of oxygen from the outlet 16 of flowmeter 10 either to a cannula 50 or to nebulizer tube 60.

In the embodiment shown, indicia 32 includes the letter “O”, while indicia 31 includes the letters “XYGE” and indicia 34 includes the letter “N”, such that oxygen is spelled in vertical orientation when the flow of oxygen is directed from the flowmeter downwardly, as shown by arrow A, into a cannula humidifying tank 52 through fitting 26 and mating fitting 54. When handle 30 is rotated 90° counterclockwise, the oxygen flow is in the direction shown by arrow B in FIG. 4 to provide an increased flow rate of oxygen controlled by knob 18 to nebulizer tube 60. Valve 20, as best seen in FIGS. 3 and 4, includes a center ball 40 which is rotatably and sealably mounted within valve body 24 in a conventional manner and includes a generally T-shaped passageway 42 with a channel 44 extending between a first end 41 and a second end 43 of ball 40. An orthogonal channel 45 of ball 40 extends from channel 44 in orthogonal relationship. Ball 40 is integrally coupled to control handle 30 to be rotated between the position shown in FIG. 3 and the position shown in FIG. 4 to provide oxygen to either outlet 27 or outlet 29. In the FIG. 3 position, oxygen flows directly downwardly through the valve into the humidifying tank 52, which includes distilled water which humidifies the oxygen and which includes an outlet 56 to which a tube 58 is coupled leading to the cannula 50 including a nose piece 55 for encircling a patient's head and inserting into the nostrils of a patient's nose during assisted breathing. The cannula 50 could also be replaced by a mask, such as mask 70 (FIG. 1), if desired.

When it is necessary to provide a nebulizing treatment to a patient, valve handle 30 is rotated counterclockwise 90° to the position shown in FIG. 4 in which the channel 45, which was sealed against the valve body 24 in the position shown in FIG. 3, now becomes the inlet for the flow of oxygen from inlet 25. Channel 44 provides a flow path at its left end, as shown by arrow B in FIG. 4, through outlet 29 into fitting 28, with the right end of channel 44 being sealed against the valve body 24 as shown. The ball valve can conventionally be made of a sealable polymeric material, such as polyvinyl chloride (PVC), polypropylene, CPVC, nylon, or other suitable polymeric material, or can be made of bronze, stainless steel, or the like. The valve conventionally includes a valve stem coupling handle 30 to ball 40 in a conventional manner. When in the position shown in FIG. 4, the oxygen flow rate typically is increased from the cannula flow rate of about 2-3 lpm to about 8 to 10 lpm through nebulizer tube 60 into a medicinal dispensing canister 64 having an outlet which is either coupled to a nebulizer breathing tube 66, including a mouth piece 68 which the patient inserts into the mouth to draw the medicament through outlet 65 in canister 64 assisted by ambient air through opening 67 in nebulizer tube 66. Alternately, a mask 70 can be attached to the outlet 65 through coupling 75 for such a treatment.

In some embodiments, it may be desirable to allow the valve handle 30 to be rotated to a third position 90° clockwise from that shown in FIG. 3 to an off position where no oxygen flows into either the cannula or the nebulizer, although, typically the flow valve 18 is simply turned off.

Thus, with the apparatus of the present invention, a patient may receive either auxiliary breathing oxygen through the cannula 50 (or a mask coupled to tube 58) or a nebulizer treatment through a nebulizing tube 66 or a mask 70. Thus, a treatment station has the ability for treating patients with either nebulizer or supplemental oxygen without the need for disconnecting and reconnecting different equipment to the outlet 16 of flowmeter 10 in each of the hospital rooms or respiratory treatment stations. The valve body 24 may include additional indicia, such as indicia 32 and 34 on the left and right side of the valve aligned with outlet 29 such that the combination of letters spell “oxygen” in the direction of flow, when the direction of flow of oxygen is that shown in FIG. 4, to assist the user in identifying the direction of diversion of the oxygen flow from flowmeter 10.

It will become apparent to those skilled in the art that various modifications to the preferred embodiment can be made. Thus, the valve 20 may include quick disconnect fittings which mate with similar fittings at the outlet of the flowmeter and inlet to the humidifying tank 52, as well as nebulizer tube 60, if desired. Other conventional threaded connections may also be employed. These and other modifications to the preferred embodiment of the invention can be made by those skilled in the art without departing from the spirit or scope of the invention as defined by the appended claims.

The invention claimed is:

1. A three-way diverter valve for supplying oxygen from a flow meter to either an assisted breathing apparatus or a nebulizer comprising:
a valve body having an inlet and a fitting attached to said inlet for coupling to an outlet of a flowmeter, and fittings coupled to said first and second outlets for coupling to one of a breathing apparatus and a nebulizer; and
wherein said ball valve includes a ball coupled to a control handle which extends from said valve body, said ball including a passageway which couples said inlet in fluid communication with said first outlet when said handle is in a first position and to said second outlet when said valve handle is rotated to a second position.

2. The valve as defined in claim 1 wherein said valve body and handle include indicia which cooperates with one another to indicate the direction of low of oxygen through said valve.

3. The valve as defined in claim 2 wherein said indicia on said valve body and handle comprises letters spelling the word “oxygen” when the valve is in at least said first position.

4. The valve as defined in claim 3 wherein said passageway is generally T-shaped.

5. The valve as defined in claim 4 wherein said valve body and ball are made of a polymeric material which is one of a group of polymeric materials including PVC, PE, CPVC, and nylon.

6. A system for supplying oxygen to one of two breathing assisting apparatus comprising:
a flowmeter having an inlet adapted to be coupled to a supply of oxygen and having an outlet for supplying oxygen at selectable flow rates;
a three-way diverter valve having a body and a control handle, said diverter valve having an inlet coupled to said outlet of said flowmeter, said diverter valve also having first and second outlets;
an oxygen breathing assist apparatus for coupling to said first outlet of said valve; and
a nebulizer treatment apparatus for coupling to said second outlet of said valve, such that oxygen from said flowmeter can be diverted to either said assisted breathing apparatus or said nebulizing apparatus by the rotation of said control handle of said diverter valve.

7. The system as defined in claim 6 wherein said diverter valve is a ball valve having a flow control ball with a generally T-shaped passageway formed in said ball.

8. The system as defined in claim 6 wherein said diverter valve includes indicia indicating the direction of oxygen flow from said inlet to one of said first and second outlets.

9. The system as defined in claim 8 wherein said control handle is raised fro the body of said valve to facilitate its use.

10. The system as defined in claim 9 wherein said control handle has a curved outer surface.

11. The system as defined in claim 10 wherein said indicia on said valve body and handle comprises letters spelling the word “oxygen” when said diverter valve is in a first position.

12. The system as defined in claim 6 and further including couplings attached to said diverter valve body to facilitate coupling to said flowmeter and to one of said breathing and nebulizing apparatus.

13. The system as defined in claim 12 wherein said breathing apparatus is a cannula.

14. A three-way diverter valve for supplying oxygen from a flowmeter to either an assisted breathing apparatus or a nebulizer comprising:
a valve body having an inlet adapted to be coupled to an outlet of a flowmeter, and first and second outlets adapted to be coupled to one of a breathing apparatus and a nebulizer; and
wherein said ball valve includes a ball coupled to a control handle, said ball including a T-shaped passageway which couples said inlet in fluid communication with said first outlet when said handle is in a first position and to said second outlet when said valve handle is rotated 90°.

15. The valve as defined in claim 14 wherein said valve body and handle include indicia which cooperates with one another to indicate the direction of flow of oxygen through said valve.

16. The valve as defined in claim 15 wherein said indicia on said valve body and handle include letters spelling the word “oxygen” when said valve is in at least said first position.

17. The valve as defined in claim 16 wherein said valve handle is raised from the body of said valve to facilitate its use.

18. The valve as defined in claim 17 wherein said valve handle has a curved outer surface.

19. The valve as defined in claim 18 wherein said diverter valve includes couplings attached to said inlet and said first and second outlets for coupling said valve to a flowmeter, a breathing assist apparatus, and to a nebulizer.

20. The valve as defined in claim 14 wherein said diverter valve includes couplings attached to said inlet and said first and second outlets for coupling said valve to a flowmeter, a breathing assist apparatus, and to a nebulizer, respectively.
